(19) United States
(12) Patent Application Publication
(10) Pub. No.: US 2022/0062484 A1
(43) Pub. Date: Mar. 3, 2022

(54) FRAGRANCE PACKAGING, CARTRIDGE INSERT, AND METHOD FOR FORMING A FRAGRANCE PACKAGING FOR A SCENT DISPENSING SYSTEM

(71) Applicant: Etienne Iliffe-Moon, Menlo Park, CA (US)

(72) Inventor: Etienne Iliffe-Moon, Menlo Park, CA (US)

(21) Appl. No.: 17/004,043

(22) Filed: Aug. 27, 2020

(51) Int. Cl. A61L 9/12; A61L 9/04

(52) U.S. Cl. CPC A61L 9/12; A61L 2209/133; A61L 9/042

(57) ABSTRACT

Embodiments generally relate to a fragrance packaging for a scent dispensing system. In particular, embodiments relate to a toroidal shaped fragrance packaging for a scent dispensing system. A fragrance packaging comprises a body with an anterior portion and a posterior portion. The body forms a shell having an inlet at the anterior portion, an outlet at the posterior portion. The shell substantially encloses an at least one fragrance chamber in fluid connection with the inlet and the outlet. The inlet and the outlet comprise a plurality of apertures. The at least one fragrance chamber comprises at least one scent medium, which is unable to pass from the fragrance chamber through the inlet or outlet.

### FIELD

Embodiments generally relate to a fragrance packaging for a scent dispensing system. In particular, embodiments relate to a toroidal shaped fragrance packaging for a scent dispensing system.

### BACKGROUND

The human body is affected by its sense of smell. Leveraging a person's sense of smell can cause both physiological and psychological effects. It can also affect the sensory system as a whole. A smell can alter a person's emotion, mood, memory, and perception. And, in turn, how a person perceives a smell can be influenced by how the smell or fragrance is packaged.

Many industries, including the automotive industry, have incorporated a fragrance experience into their products. Typically, fragrances are blends of chemical compounds that are derived from natural or synthetic sources. These are typically presented to consumers as liquid-based fragrances such as oils.

However, many people have a negative perception about the sources and ingredients used in products like fragrances, perfumes, and air fresheners. Many users of fragrance products, including those who seek products for use in vehicles, desire more natural and organic fragrances that promote or encourage health and wellness. Hence, there may be a desire for an improved fragrance packaging for a scent dispensing system that conveniently, effectively, and practically delivers natural and organic scent media.

### SUMMARY

Embodiments in the present disclosure relate to a fragrance package or packaging for a scent dispensing system. According to an embodiment, the packaging may comprise a body with an anterior portion and a posterior portion. The body forms a shell having an inlet at the anterior portion and an outlet at the posterior portion. The shell substantially encloses at least one fragrance chamber that is in fluid connection with the inlet and outlet. The inlet and the outlet comprise a plurality of apertures that allow air to pass between the interior and exterior of the body. Each fragrance chamber may comprise at least one scent medium, which is unable to pass from the chamber through the inlet or outlet.

Dispensing a fragrance in its natural form is difficult and poses several issues. Some fragrance ingredients exist in their natural form with a high level of potency such that they can be used directly as a fragrance. These include botanicals such as lavender or rose petals. Other ingredients require extraction processes and require significant concentrations, such as rose oil, to be potent for use as a fragrance. Additional examples of extracts include absolute, essential oils, and tincture. These extracts may need to be packaged or provided on a substrate or carrier that would be compatible with the natural form of the ingredients.

Many fragrances can be derived from a single ingredient (e.g. jasmine, rose, lavender, pinyon pine, etc.) but often fragrances are blended to create more complex or nuanced aromas. Blending natural and synthetic ingredients—while still retaining the natural or solid form of the ingredient—is not always possible. So, packaging a fragrance with ingredients in their natural form or with natural extracts may preserve the ingredients or keep them fresh to maintain their potency.

Packaging fragrance ingredients, elements, materials, or other scent media into a sub-container, bag, or vessel allows for these potent ingredients to be conveniently, effectively, and practically introduced into a scent dispensing system without contaminating a fragrance container or chamber of the system. It also allows for easy and convenient removal of depleted scent media from the system. And it may allow for visual or olfactory inspection of the ingredients by a user.

According to another embodiment, the shell of the fragrance packaging is toroidal, forming an at least one channel that extends or passes from the anterior portion of the body to the posterior portion of the body. A toroid shape may allow for greater visual inspection of the contents of the packaging. It also may allow for channels to form in the body of the package to better fit a chamber in a scent dispensing system.

The at least one channel may be formed to allow for a valve arrangement to pass through the at least one channel. A valve arrangement may, for example, allow the package to work in environmental conditions that may otherwise cause a scent leakage. It may also allow for the controlled release of the fragrance into a vehicle cabin by preserving or prolonging scent media. A valve arrangement may control airflow through the packaging. The packaging may comprise multiple valve arrangements associated with multiple fragrance chambers and multiple airflow sources for the multiple chambers. Having multiple airflow sources may allow for setting independent airflows for one or more chambers.

A valve arrangement may comprise an inlet sealing member and an outlet sealing member. These sealing members may be in connection so that the position of the inlet sealing member corresponds with the position of the outlet sealing member. This connection allows for the instant and simultaneous adjustment of the inlet and outlet sealing members. This connection may allow for a simpler mechanical, electrical, or computational structure to open both valves simultaneously. The channel formed in the toroid shell allows for this valve arrangement to be physically connected either by mechanics, electrics or otherwise.

The fragrance packaging may include at least one empty fragrance chamber for generating a scent-neutral airflow component. A scent-neutral component may help flush stray scent molecules from the device without introducing additional scents. The scent-neutral component may be used to accelerate the dissipation of a scent present in the environment, whether or not it was initially dispensed by the packaging or scent dispensing system. It may also be used in conjunction with the delivery of a scent from another fragrance chamber to augment the scent, potentially by diluting a scent and moving or dissipating the scent into the surrounding ambient air. This may help avoid olfactory adaptation or olfactory fatigue, which is a phenomenon that results in a temporary inability to distinguish an odor after prolonged exposure to it.

Additionally, air may be ventilated through an empty chamber without scent media. This ventilation may be activated alone or in combination with airflow through one or more chambers. The ventilation may purge or clean a system of any residual scent, increase the aeration of the scent stream, or increase the airflow before, during, or after a scent release. This ventilation carries the scent away from the device and increases scent diffusion and dissipation.

In another embodiment, the valve arrangement comprises a sealing member arranged to seal a portion of at least one of the inlet or outlet of the shell, wherein the sealing member restricts airflow through the at least one fragrance chamber. A valve or sealing member that provides a physical seal can prevent leakage of scent molecules and halt or block airflow. If a package has more than one chamber, a further chamber it may have a valve that can be controlled independently, in combination, or in parallel with other chambers. Additionally, by localizing the valves with respect to the packaging, sealed connections to other components might not be required. In automotive applications, the environmental conditions can be such that scent dispensing systems may leak scents. This can be improved by sealing the fragrance package with valves that provide a physical seal that turn off, halt, or block the airflow. The valve arrangement may have inlet valves at the shell inlet and outlet valves at the shell outlet.

Sealing members might not be 100% open or closed but may have variable positions such as 25%, 50%, 75%, or 100% open, or any value between 0% and 100%. Sealing members are arranged so that airflow is enabled to flow from the exterior of the body through the channel and then return to the exterior when the at least one inlet or outlet sealing member are in the open position. Airflow is disabled or stopped through the channel when at least one of the inlet or outlet sealing member is in the closed position.

Sealing members should be arranged so that the scent media cannot interfere with the operation of the valves as they transition from an open to a closed position. This may be important for environments that experience extreme effects, such as within an automobile or other vehicle. The scent media in an automobile may experience forces in all directions, making it more likely for the media to escape than in a stationary environment. Further, the extreme temperature and humidity effects that may be present in an environment like an automobile may change the structure of the scent media (i.e. separation of the scent formula, evaporating the scent media, or making it more pliable). The fragrance packaging described here better isolates the scent media from these effects to preserve the integrity of the scent media. It also contains any results of these environmental effects and allows for better control of the release of a fragrance or scent into the environment.

The scent medium may be a natural ingredient or a synthetic ingredient. In some embodiments, the scent medium may be a blend of both natural and synthetic ingredients. Natural fragrances are defined by the International Fragrance Association (IFRA) as complex compounds derived from natural aromatics that are extracted from plants without altering the chemical structure. The ingredients in natural fragrances may be liquid-based. They may also be essential oils, oleoresins, distillates, fractions, concretes, absolutes, or isolates. The ingredients of a natural fragrance can come from any natural source. Natural ingredients may be perceived by a user or consumer to be healthier and more ecologically friendly than those made from synthetic or artificial ingredients. Natural ingredients may be extracted by various methods, including steam distillation, cold pressing, expeller pressing, refining, RBD (refining, bleaching, and deodorizing), or dehydration.

Synthetic fragrances and their ingredients may be designed to mimic natural smells. These ingredients may last longer and be more potent than their natural counterparts. Synthetic ingredients may cost less than natural ingredients. Synthetic ingredients may also be able to produce fragrances or scents that do not exist naturally or only exist naturally as a blend of multiple ingredients. In some circumstances, specific synthetic ingredients may have a lower impact (e.g. environmental, ethical, health, etc.) than natural ingredients.

The natural ingredient or the synthetic ingredient may disposed on, infused with, or otherwise connected to or linked with a substrate. The substrate may comprise a perforated or grill structure. This may allow for increased airflow to pass through the chamber as well as for a greater surface area of the substrate to be exposed to the passing air. The substrate may be made of at least one element of the group of a gel-based substrate, a polymer-based substrate, a solid material, a bead-like material, and a liquid absorbing or holding material. A scent medium made partially or entirely of a solid-state substance or format may better withstand extreme environmental conditions, such as extreme temperature and humidity.

In another embodiment, the substrate may be an organic material. The organic material may be one of the group of paper, fabric, leaf, petal, seed, seed pod, flower, and bark. Disposing a scent ingredient on an organic substrate may allow the packing to be more ecologically friendly, disposable, and biodegradable.

A fragrance packaging may comprise an additional media including at least one element of the group of an anti-bacterial, a disinfectant, an odor treatment, or an odor neutralization media. These media may act to remove odors from the atmosphere or environment outside the scent dispensing system. They may also act to cleanse or deodorize any system connected to or comprising the packaging. It may also decontaminate valves or other components in connection with the packaging that may become contaminated with scent molecules. Decontaminating the system connected to the package may improve the operation of the system's mechanical components by removing gunk or other debris that interfering with the system's operation. This may prolong the life of the system or the packaging and prevent the unwanted leakage of scents from the suboptimal operation of the system, particularly its valves. Additionally, deodorizing the system or the surrounding atmosphere and environment may allow for the scent that is dispensed from the package to be more readily perceived. This may result in the overall reduction in scent delivered for a user to perceive the scent, prolonging the life of the packaging and the scent media contained within it.

The fragrance packaging may be comprised of a polymer or glass. Polymers and glass are readily available materials that may be molded into complex shapes, including a toroid. The polymer or glass may be partially or completely transparent. These materials may form a plurality of windows in the shell, which allows the user to better perceive and understand the quality of the fragrance packaged inside. This also may allow a user or consumer to peer inside the packaging and inspect its fragrance contents. This may mean that portions of the packing are opaque so long as windows or other generally transparent portions of the shell allow the scent media to be identified.

The previously described fragrance packaging may have a substantially transparent shell. The fragrance, whether it is a single ingredient or a blend, may be packaged so that the ingredients look natural and are perceived to be natural by a user or consumer. A package that is substantially transparent may allow for fragrance ingredients to be visible, partially visible, or packaged such that the fragrance packaging can be opened and viewed by the consumer. Substantial transparency might not be 100% but may be a such as 25%, 50%, 75%, or 100% transparent, or any value between 0% and 100% so that the contents of the fragrance chamber are identifiable or distinguishable.

In another embodiment, the shell is comprised of a porous material. It may be comprised of perforated material or have a mesh form. The material form would be such to allow airflow through or around the ingredients (e.g. be perforated, micro-perforated, have a grill structure, etc.). For example, the porous material may be paper, fabric, or a polymer. Paper may allow for the packaging to be more ecologically friendly, disposable, and biodegradable. Other porous materials include filter paper, perforated paper, micro-perforated polymers, and woven or non-woven fabrics.

The use of fabric allows for air to pass through the material and circulate more freely through the fragrance chamber. Porous fabrics, such as woven or knitted performance fabrics or meshes (e.g. with bi-axial or tri-axial structures), are breathable and allow air to freely pass through, due to either a loosely woven texture or structure produced from a synthetic or natural yarn. Non-woven fabrics are generally fabric-like materials made from staple fiber and long fibers that are bonded together by chemical, mechanical, heat, or solvent treatments. Non-woven fabrics are typically flat or tufted porous sheets. The term is used in the textile manufacturing industry to denote fabrics, such as felt, which are neither woven nor knitted. External treatment of fabric can drastically alter or change a material's absorption ability for air to pass through freely. Therefore, the treatment of the fabric may be such that it remains porous to retain its breathability.

The porous material may include a ceramic, a metal, and a polymer. Non-porous materials retard or slow fluid absorption, meaning air is not able to pass freely through tightly bonded non-porous materials. However, hard materials may be porous by their nature or by their construction. For example, although both granite and sandstone are natural stones or rocks, sunstone is known to be very porous, especially compared to granite. Likewise, raw concrete is much more porous than concrete that is polished or sealed with a water-repellent substance.

In another embodiment, a fragrance packaging may comprise a body with an anterior portion and a posterior portion, the body adapted to fit a cartridge chamber in a scent dispensing system. The body may comprise a porous material. It may form a toroidal shell wherein an at least one channel is formed through the body parallel to an axis extending from the anterior portion to the posterior portion. The toroid shell may substantially enclose an at least one fragrance chamber comprising at least one scent medium. The scent medium should be unable to pass from the at least one fragrance chamber to the cartridge chamber. The scent medium should be unable to interfere with the operation of the scent dispensing system. Such as by interfering with a transition of the at least one inlet or outlet sealing member between the open position and the closed position.

Additionally, there may be a method for packaging an at least one scent medium. The method may comprise forming a shell having an anterior portion and a posterior portion, wherein an interior of the shell substantially comprises an at least one fragrance chamber comprising the at least one scent medium. The method may further comprise forming an inlet at the anterior portion and an outlet the a posterior portion, wherein the inlet and the outlet comprise a plurality of apertures, and wherein the fragrance chamber is in fluid connection with the inlet and the outlet. The scent medium should be unable to pass from the fragrance chamber through the inlet or outlet.

The previously described fragrance packaging may be inserted into a cartridge of a scent dispensing system. The system may have at least one airflow source configured to generate airflow through at least one chamber of the cartridge. The system may have a controller coupled to the at least one airflow source and is configured to control the airflow source and one or more valves to generate a fragrance or scent mixture outside of the system from an at least one airflow through the fragrance packaging inserted into the cartridge. A scent dispensing system may be part of a larger or wider air circulation system, such as a heating, ventilation, and air conditioning (HVAC) system where the airflow source is external to the system. The system may be configured to operate the valves to control the dispersal of a scent from the fragrance packaging.

The fragrance packaging is removably insertable into the previously described scent dispensing system. The removability of the package allows it to be replaced allowing for new scent experiences without the replacement of the entire scent dispensing system.

A vehicle may comprise the previously described scent dispensing system with the fragrance packaging. A vehicle may be any type of land-, sea-, and air-based vehicle or transportation system. The fragrance packaging, as described, may be particularly well suited to the extreme environments present in many land-, sea-, and air-based vehicles.

The previously described scent dispensing system and fragrance packaging may be integrated into an HVAC system of the previously described vehicle. Integration into existing vehicle systems allows for the system to control the dispersal of a scent using existing airflow systems, as well as allowing the scent to be adjusted using additional elements like heating, ventilation, humidifying or dehumidifying, and air conditioning elements. This may prolong a scent, disperse a scent, dissipate, neutralize, or remove a scent (such that it can no longer be smelled), adjust the character of a scent, or allow multiple scents to better intermix, creating new scent experiences. For example, a scent dispensing system may humidify a scented airflow (such as a tropical scent) before or as it is released into a cabin of a vehicle, creating a richer and more immersive scent experience by mimicking a tropical environment.

The previously described scent dispensing system in a vehicle is controlled by one or more elements of the group of a vehicle operation status component (i.e. the vehicle's onboard computer that provides basic/core vehicle system info like speed, gear selection, lighting, etc.) a navigation or traffic component, an infotainment component, communication component, weather information, safety monitoring component, and an autonomous driving system component. This allows for the system to be integrated with other external systems that can adjust and inform the perception of the smell released by the fragrance packaging. For instance, using weather information could allow the system to attune scent based on humidity, temperature, or other aspects of the weather. Also, using infotainment could allow the scent to be adjusted to coincide with other sensory effects, such as music or video, heightening a users' perception of a scent.

### BRIEF DESCRIPTION OF THE DRAWINGS

Some embodiments of apparatuses and systems will be described in the following only by way of example and with reference to the accompanying figures, in which:

FIGS. 1A, 1B, 1C, and 1D show a view of an embodiment of a fragrance packaging.

FIGS. 2A, 2B, and 2C show a view of an embodiment of the fragrance packaging with a plurality of channels formed by the toroidal shell.

FIGS. 3A and 3B show a view of an alternative embodiment of the fragrance packaging with a valve apparatus.

FIG. 4 shows simplified views of substrate embodiments of the scent media.

FIG. 5 shows a block diagram of a decision tree embodiment for different types of scent media.

FIG. 6 shows a block diagram of an embodiment of a method for packaging a fragrance.

### DETAILED DESCRIPTION

Various embodiments will now be described more fully with reference to the accompanying drawings in which some embodiments are illustrated. The thicknesses of lines, layers, and/or regions in the figures may be exaggerated for clarity.

Accordingly, while further embodiments are capable of various modifications and alternative forms, some particular embodiments thereof are shown in the figures and will subsequently be described in detail. However, this detailed description does not limit further embodiments to the particular forms described. Further embodiments may cover all modifications, equivalents, and alternatives falling within the scope of the disclosure. Same or like numbers refer to like or similar elements throughout the description of the figures, which may be implemented identically or in modified form when compared to one another while providing for the same or similar functionality.

It will be understood that when an element is referred to as being “connected” or “coupled” to another element, the elements may be directly connected or coupled via one or more intervening elements. If two elements A and B are combined using an “or”, this is to be understood to disclose all possible combinations (i.e. only A, only B, as well as A and B) if not explicitly or implicitly defined otherwise. An alternative wording for the same combinations is “at least one of A and B” or “A and/or B”. The same applies, mutatis mutandis, for combinations of more than two elements.

The terminology used herein for the purpose of describing particular embodiments is not intended to be limiting for further embodiments. Whenever a singular form such as “a,” “an” and “the” is used—and using only a single element is neither explicitly or implicitly defined as being mandatory—further embodiments may also use plural elements to implement the same functionality. Likewise, when a functionality is subsequently described as being implemented using multiple elements, further embodiments may implement the same functionality using a single element or processing entity. It will be further understood that the terms “comprises,” “comprising,” “includes” and/or “including,” when used specify the presence of the stated features, integers, steps, operations, processes, acts, elements, and/or components but do not preclude the presence or addition of one or more other features, integers, steps, operations, processes, acts, elements, components and/or any group thereof.

Unless otherwise defined, all terms (including technical and scientific terms) are used herein in their ordinary meaning of the art to which the embodiments belong.

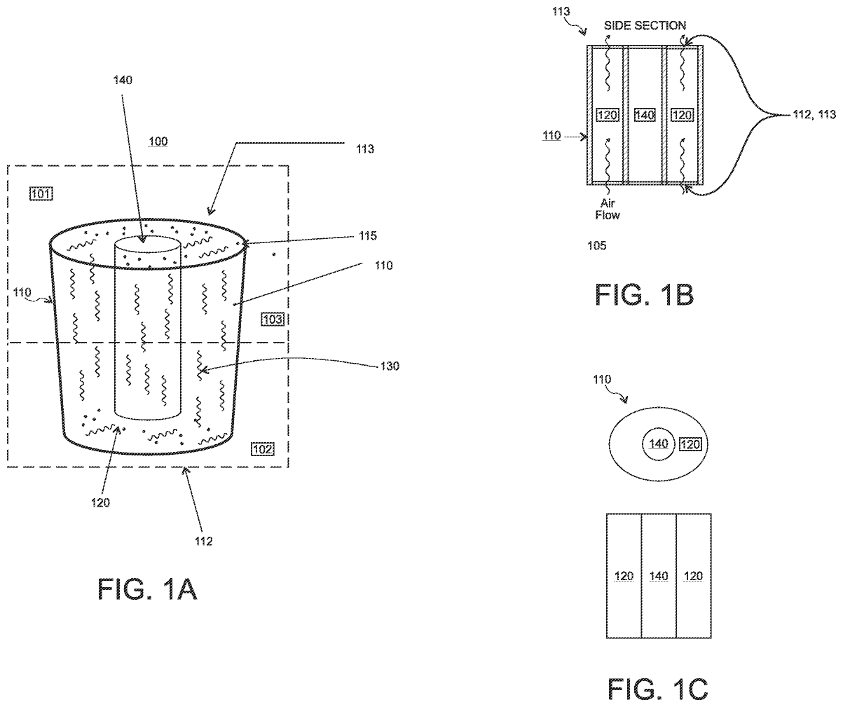

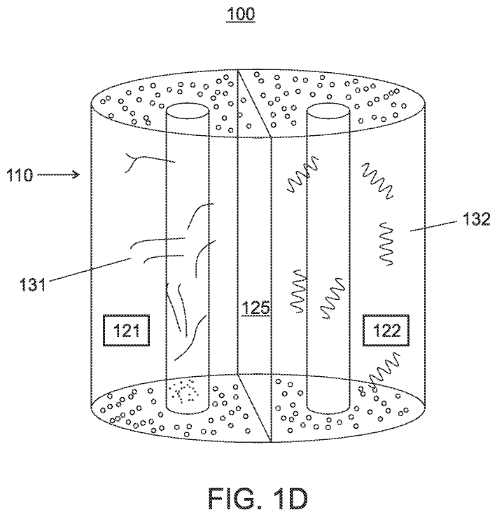

FIGS. 1A, 1B, 1C, and 1D show a view of an embodiment of a fragrance package or packaging. FIG. 1A shows the elements of a fragrance packaging 100 for a scent dispensing system. The fragrance packaging comprises a body 101 with an anterior portion 102 and a posterior portion 103. The body 101 forming a shell 110 having an inlet 112 at the anterior portion 102, an outlet 113 at the posterior portion 103, and the shell 110 substantially enclosing an at least one fragrance chamber 120. The fragrance chamber 120 is in fluid connection with the inlet 112 and the outlet 113. The inlet and outlet comprise a plurality of apertures 115. The at least one fragrance chamber 120 comprises at least one scent medium 130. The at least one scent medium 130 is unable to pass from the at least one fragrance chamber 120 through the inlet 112 or the outlet 113.

The fragrance packaging 100 may be produced using any conventional manufacturing technique, including casting and molding, machining, joining, shearing, and forming. It may also be formed using additive manufacturing systems and techniques, often referred to as three-dimensional (3D) fabrication or 3D printing. The shell 110 may be made from metals, wood or paper, polymers, or any other material that can sufficiently store scent media without leakage. It may also be made from a combination of materials or a composite material. The use of biodegradable materials, such as wood or paper, may aid with the disposal of the packaging 100 in an environmentally friendly way.

FIG. 1A further shows the fragrance packaging 100 wherein the scent medium 130 is a natural ingredient or a synthetic ingredient. Natural fragrances as defined by the International Fragrance Association (IFRA) are complex compounds derived from natural aromatics that are extracted from plants without altering the chemical structure. Natural ingredients may be essential oils, oleoresins, distillates, fractions, concretes, absolutes, or isolates and may come from any natural source. Synthetic fragrances may be designed to mimic natural smells as well as last longer and be more potent. Synthetic ingredients may cost less than natural ingredients. Synthetic ingredients may also be able to produce fragrances or scents that do not exist naturally or only exist naturally as a blend of multiple ingredients.

Scent media 130 may include other media for aerosolization, diffusion, or dispersion from the packaging 100, such as media to remove an odor or scent from the environment or otherwise cleanse, filter, or disinfect the atmosphere or environment outside the packaging 100 or scent dispensing system. The shell 110 may comprise an at least one chamber 120 containing at least one element of the group of an anti-bacterial, a disinfectant, an odor treatment, or an odor neutralization media.

FIG. 1B shows a cross-sectional view of the embodiment where air 105 flows from the inlet 112 of the shell 110 through the fragrance chamber 120 to the outlet 113 of the shell. The inlet and outlet 112, 113 may have a perforated or grill structure that allows airflow 105 to pass through inlet and outlet 112, 113 but does not allow the scent media 130 to escape or leak from the inlet or outlet 112, 113 of the shell 110. In some embodiments, inlet and outlet 112, 113 may comprise substantially the entire anterior and posterior portions 102, 103. In these embodiments, the shell 110 may consist essentially of a perforated or grill structure with a plurality of apertures 115. Airflow 105 may be produced by one or more fans, pumps, compressors, or any devices configured to move air.

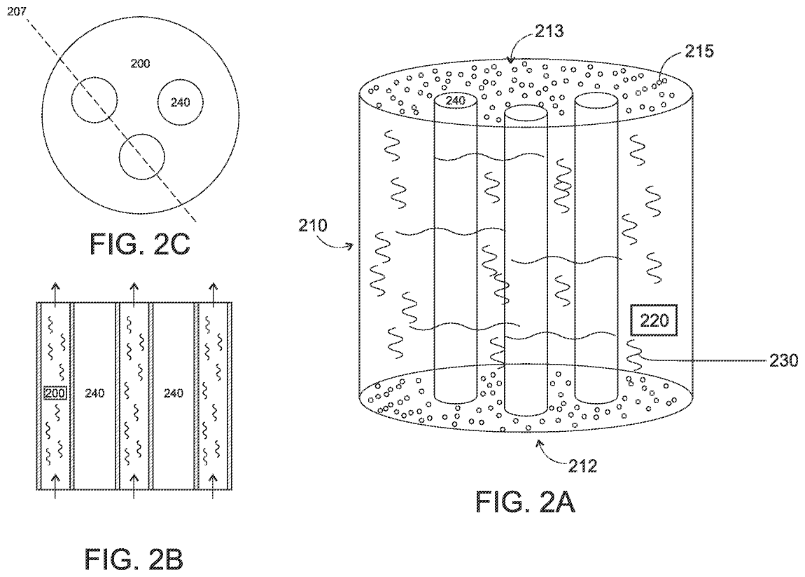

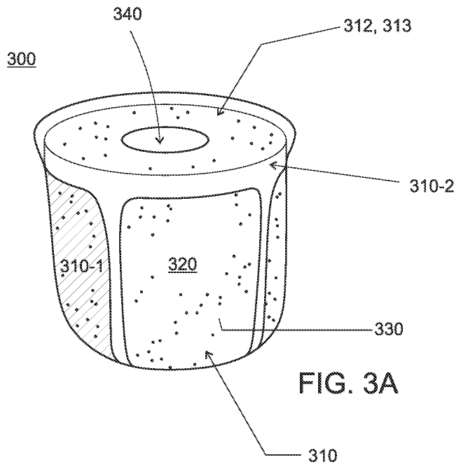

The shell 110 may be configured to isolate scent 130 or other media from any moving components (e.g. a valve arrangement 250, as shown in FIG. 2B, passing through the channel 140). For example, the apertures 115 of the inlet and the outlet may be formed so that they prohibit the scent media 130 from leaving the fragrance chamber 120. Containing and isolating the scent media 130 prevents leakage of residue and the possibility that residue can interfere, gum up, or contaminate moving or running surfaces causing any mechanical movement to become slow, sticky, or otherwise prevent smooth mechanical movement.

FIG. 1D shows an embodiment wherein the fragrance packaging 100 comprises a plurality of fragrance chambers 121, 122. The figure shows the shell 110 divided by an internal structure 225 or wall to separate the fragrance chambers 121, 122 and their scent media 131, 132. The fragrance chambers 121, 122 may each comprise separate scent media 131,132.

In some embodiments, the shell 110 may comprise at least one empty fragrance chamber 130 for generating a scent-neutral airflow component. Passing air through at least one empty chamber 130 may purge or clean the system of any residual scent, increase the aeration of the scent stream, or increase the airflow (e.g. before, during, or after a scent release) to carry the scent away from the fragrance packaging or scent dispensing system and increase scent diffusion and dissipation. The ventilation of air through an empty chamber 120 may be activated alone or in combination with airflow through one or more additional chambers 120.

FIGS. 1A and 1B further show the fragrance packaging wherein the shell is toroidal forming an at least one channel 140 that extends from the anterior portion 102 of the body to the posterior portion 103 of the body. FIG. 1C shows an overhead view of the fragrance packaging 100. Particularly, FIG. 1C shows the toroid structure. In geometry, a torus is a surface of revolution generated by revolving a circle in three-dimensional space about an axis that is coplanar with the circle. If the revolved shape is not a torus, as shown in the figures, the shape is called a toroid. Also, the revolution might not be perfectly circular. For example, a toroidal polyhedron can be constructed to approximate a torus. An ordinary torus has a genus of one meaning that it has one hole or channel that extends through it. Higher genus toroids or tori, sometimes called n-holed or n-fold tori, have channels equal to the number of the genus. A 2-genus toroid is sometimes called a double toroid or torus and a 3-genus toroid is sometimes called a triple toroid or torus.

FIGS. 2A, 2B, and 2C show a view of an embodiment of the fragrance packaging with a plurality of channels formed by the toroidal shell. Specifically, FIG. 2A shows a view of an embodiment of the fragrance packaging 200 with three channels formed by the 3-holed toroidal shell 210. FIG. 2C shows an overhead view of the 3-holed toroidal shell 210 of the fragrance packaging 200. And FIG. 2B shows a cross-sectional view of the 3-holed toroidal shell 210 along the dividing line 207.

FIG. 2A further shows a shell 210 having an inlet 212 and an outlet 213. And the shell 210 substantially enclosing an at least one fragrance chamber 220. The fragrance chamber 220 is in fluid connection with the inlet 212 and the outlet 213. The inlet and outlet comprise a plurality of apertures 215. The at least one fragrance chamber 220 comprises at least one scent medium 230.

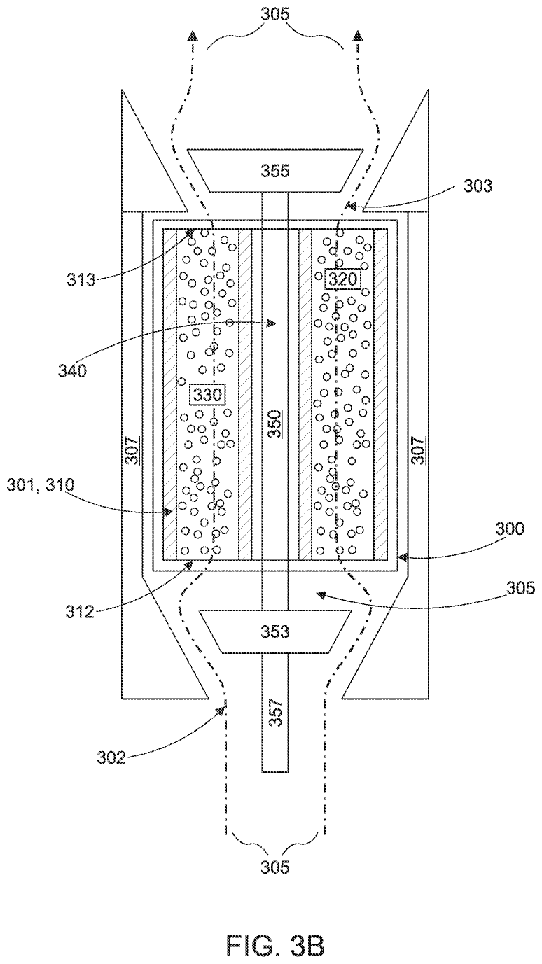

The FIGS. 1A, 1B, 2A, 2B, and 2C also show at least one channel that is formed to allow for a valve arrangement 350 to pass through the at least one channel 140. As shown in FIG. 3B, the valve arrangement 350 may comprise a sealing member arranged to seal a portion of at least one of the inlet or outlet of the shell, wherein the sealing member restricts an airflow through the at least one fragrance chamber.

FIG. 3A shows the fragrance packaging 300 wherein a shell 310 is comprised of a polymer or a glass. The polymer or the glass may also form a plurality of windows 310-1 in the shell 310. Additionally, the shell 310 or a window of the shell 310-1 may be comprised of a porous material, such as paper or a polymer. Other porous materials include filter paper, perforated paper, perforated polymers, and woven or non-woven fabrics. Perforated may mean any material that comprises a series of small or very small (micro) holes. In some embodiments, the holes may be arranged at 2, 5, 10, 20, or more holes per centimeter. The porous material may also be at least a ceramic, a metal, a polymer, and a natural fiber or synthetic fiber. Natural materials (such as cotton, silk, and wool) are made of animal or plant-based fibers. Synthetic fibers are man-made and produced entirely from chemicals to create materials like polyester, rayon, acrylic, and many others. The shell 310 may also be a micro-perforated polymer fabricated sheet material or a thin-wall injection molded material with in-molded perforations. The shell 310 may comprise multiple structures such as an outer frame 310-2 that provides structure and a window, mesh, screen, or other element 310-1 that is placed between the frame 310-2. This window or screen 310-1 may sit inside the frame 310-2, be connected to the frame, or otherwise integrated or attached with the frame so that it forms a toroidal shape.

The shell 310 may be substantially transparent. Transparent means having the property of transmitting rays of light through its substance so that bodies situated beyond or behind can be distinctly seen. A transparent shell 310 may transmit light without appreciable scattering—similar to window glass—so that scent media 330 in the fragrance chamber 320 are distinguishable.

FIG. 3B shows a view of an alternative embodiment of the fragrance packaging 300 (shown by the dotted line) in a cartridge 307 with a valve arrangement 350. A cartridge 307 of a scent dispensing system may have a cartridge chamber 305 in which the body 301 of the fragrance packaging may be removably inserted. The cartridge chamber 305 may have an inlet 302 and an outlet 303. A removable fragrance packaging 300 allows for the package to be replaced enabling new scent experiences without the replacement of the entire scent dispensing system. When the body 301 of the fragrance packaging 300 is inserted into the cartridge chamber 305, it might not require any physical coupling to the cartridge 307 or valve arrangement 305 to operate. In some embodiments, the valve arrangement 350 may be part of or included with the fragrance packaging 300 and also removably insertable into cartridge chamber 305 of the scent dispensing system.

A valve arrangement 350 may be any element that regulates, directs, or controls the flow of a fluid, namely air, by opening, closing, or partially obstructing the flow of a fluid through the inlet 312 or outlet 313 of the fragrance chamber 305 or the inlet 302 and outlet 303 of the cartridge chamber 305. Valves of the valve arrangement 350 may be a linear motion valve, a rotary motion valve, a quarter-turn valve, a sliding valve, or any combination or mechanism that sufficiently seals the fragrance package 300. In an open configuration, a valve arrangement 350 allows fluid, such as air, to flow in a direction from higher pressure to lower pressure. In a closed configuration, a valve arrangement works to prevent the entry of fluid into the fragrance chamber 320 or the cartridge chamber 305 and to prevent the escape of scent molecules from the shell 310. Valves may be implemented using one or more poppet valves 353, 355, which use disks or sealing members that seal an opening in a seating. The disc may be attached to a rod 357 and two discs can be attached to a single rod or shaft, forming a connected arrangement or apparatus that allows for simultaneously sealing/opening the inlet 312 and the outlet 313 of the shell 310 or the inlet 302 and outlet 303 of the cartridge chamber 305. The valve arrangement 350 may be actuated or moved by an actuator. The connection 357 between inlet and outlet valves may also be a screw, a belt, an electrical connection, or any connection that links the valves. It is not required to have a set of valves for the inlet and outlet. Inlet and outlet adjacent valves—such as those adjacent to a chamber into which the fragrance packaging is inserted or to the inlet and outlet of a scent dispensing system—may be used. These adjacent valves may still pass through the channel 340 of the packaging 300. Other mechanisms may also be used to prevent the leakage of scent media, such as positive air pressure at the inlet 312 of the shell 310 and a filter, grate, mesh, or another containment device that allows air to pass through the fragrance chamber 320 but not the scent media 330.

The corresponding positions of the sealing members or valves 353, 355 might not be identical. Operating conditions and manufacturing tolerances may allow for some inconsistency in the valve positions. The sealing members 353, 355 may be composed of a rubber or elastomeric material with enough compliance to take up the likely manufacturing tolerance and provide a sufficient seal. These materials may also reduce noise from the engagement of the valves.

The valve arrangement 350 may be oriented at the inlet 312 and outlet 313 of the shell 310. In the embodiment FIG. 3B, the shell 310 comprises inlet 312 and outlet 313 with apertures 315. The inlet 312 and outlet 313 may be constructed in any manner that allows air to pass through the fragrance chamber 320. For example, inlet 312 and outlet 313 may be constructed as injection ports, slits, funnels, sieves, other openings, or a combination of opening types. At least one of a pair of inlet and outlet valves 353, 355 in a valve arrangement 350 may float enough to accommodate potential manufacturing tolerances, and use a spring (e.g. a metal spring or plastic molded feature, etc.) or similar mechanical force to engage the floating valve. This allows for one of the valves to be actuated and for the actuated valve to dictate the position (i.e. open or closed) of the floating valve.

FIG. 3A also shows a fragrance packaging 300 for a scent dispensing system. The packaging comprises a body 301 with an anterior portion and a posterior portion, wherein the body 301 is adapted to fit a cartridge chamber 307 in the scent dispensing system. The body 301 comprises a porous material and forms a toroid shell 310 wherein an at least one channel 340 is formed through the body 301 parallel to an axis extending from the anterior portion to the posterior portion. The toroid shell 310 substantially encloses an at least one fragrance chamber 320, which comprises the at least one scent medium 330. The at least one scent medium 330 is unable to pass from the at least one fragrance chamber 320 to the cartridge chamber 307.

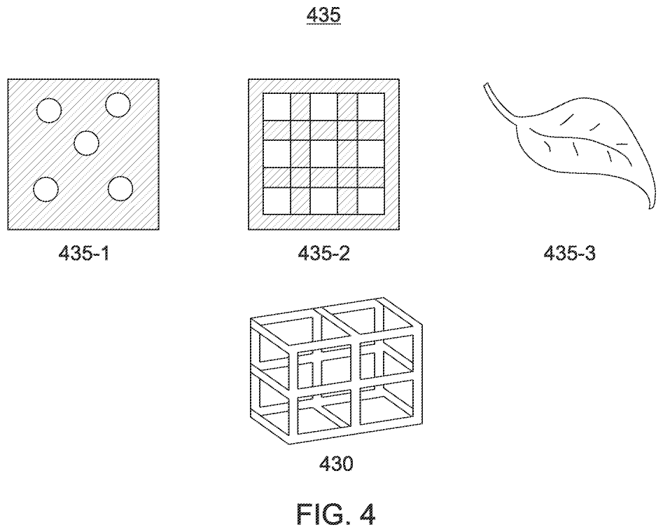

FIG. 4 shows simplified views of substrate embodiments of the scent media. In another embodiment, the natural ingredient or the synthetic ingredient is disposed on a substrate 435. The substrate of the fragrance packaging 100, 200, 300 may be gel-based, polymer-based, a solid material, a bead-like material, and a liquid absorbing or holding material. Additionally, a scent can be mixed in with a 3D printing substance to form a printing filament. The filament can then be printed to form a 3D printed scent media 430. Another alternative would be to print a 3D matrix or from a filament that comprises a scent absorbing material, then introducing the scent to the printed structure to form a scent media 430. In one embodiment, the substrate 435 comprises a perforated 435-1 or grill structure 435-1.

The substrate 435 may also be an organic material 435-3. Organic material is any material that relates or is derived from living matter. The organic material 435-A may be at least one of the group of paper, fabric, leaf, petal, seed, seed pod, flower, or bark.

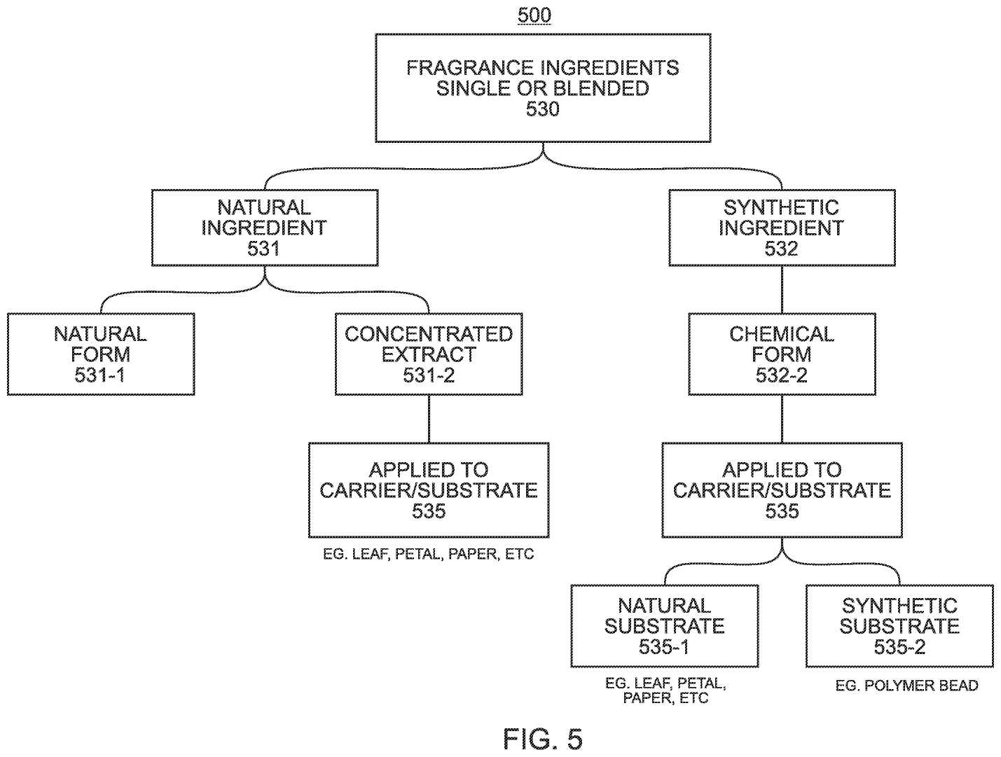

FIG. 5 shows a block diagram of a decision tree embodiment for different types of scent media. A scent media 530 may be a natural ingredient 531 or a synthetic ingredient 532. A natural ingredient 531 may be packaged in its natural form 531-1 or an extract of a natural ingredient 531-2. That extract 531-2 may be applied to a substrate 535, likely a natural substrate 535-2. A synthetic ingredient 532 may be packaged in a solid form or, if in a chemical form 532-2 applied to a substrate 535. That substrate may be natural 535-1 or synthetic 535-2.

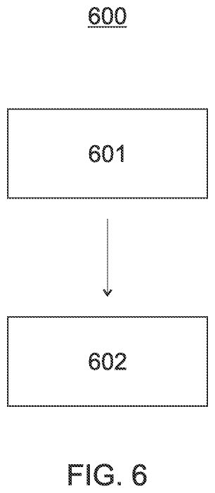

FIG. 6 shows a block diagram of an embodiment of a method 600 for packaging an at least one scent medium for a scent dispensing system. The method comprises forming 601 a shell having an anterior portion and a posterior portion, wherein an interior of the shell substantially comprises an at least one fragrance chamber comprising the at least one scent medium. The method further comprises forming 602 an inlet at the anterior portion and an outlet at the posterior portion. The inlet and the outlet comprise a plurality of apertures and the fragrance chamber is in fluid connection with the inlet and the outlet. The at least one scent medium is unable to pass from the fragrance chamber through the inlet or the outlet.

In another embodiment, the a method of packaging an at least one scent medium may comprise forming a shell having an anterior portion and a posterior portion along a first axis and a left lateral portion and a lateral right portion along a second, transverse axis. The shell comprising a porous material and wherein an interior of the shell substantially comprises an at least one fragrance chamber. The fragrance chamber comprising the at least one scent medium. The method further comprising joining the left and right lateral portions to form a toroid or a sleeve.

The fragrance packaging 100 may be replaceable. The term replaceable means that the packaging may be conveniently added to or removed from the scent dispensing system according to a user's or customer's needs. A replaceable fragrance packaging may be interchanged with different packaging (e.g. comprising different scent media or different embodiments as described in this application). A different fragrance packaging added to the system may serve a different function depending on its scent media or configuration. Further, a producer of fragrance packaging may be able to tailor several different versions of the fragrance packaging to provide a curated scent experience to the user. Each fragrance packaging may also comprise a method of identification so that when it is removably inserted into the system it may be identified. The identification may also comprise the packaging's configuration and contents. Additionally, replaceable nature of the packaging, it is possible for a user to purchase a scent dispensing system without a package if it does not meet their present needs and then add the package to the system when it better suits the user. When the fragrance packaging 100 is inserted into the scent dispensing system it might not require any physical coupling to the system to operate.

A fragrance packaging 100 may be disposable or recyclable. A recyclable packaging may be composed of recyclable materials that can be deposed into a user's recycling system or they may be returned to the manufacturer so that they can be refilled and reused.

A scent dispensing system may stand alone or may be supplied or sold as an “optional accessory”, a “factory installed” accessory or option, a dealer-installed accessory or an accessory installed by the customer or third party. The system may connect to a vehicle or other apparatus for power (e.g. electrical connection, wireless charging, etc.) for data that may be provided wired or wireless (e.g. Bluetooth, WIFI, etc.). The system may be located in a specifically designed location, receptacle, or port, or be in a pre-existing receptacle such as a cup holder, ashtray, armrest or glove box.

The aspects and features mentioned and described together with one or more of the previously detailed embodiments and figures, may as well be combined with one or more of the other embodiments to replace a like feature of the other embodiment or to additionally introduce the feature to the other embodiment.

What is claimed is:

1. A fragrance packaging for a scent dispensing system, the packaging comprising:
a body with an anterior portion and a posterior portion, the body forming a shell having an inlet at the anterior portion, an outlet at the posterior portion, and the shell substantially enclosing an at least one fragrance chamber,
wherein the fragrance chamber is in fluid connection with the inlet and the outlet,
wherein the inlet and the outlet comprise a plurality of apertures,
wherein the at least one fragrance chamber comprises at least one scent medium, and
wherein the at least one scent medium is unable to pass from the at least one fragrance chamber through the inlet or the outlet.

2. The fragrance packaging of claim 1 wherein the shell is toroidal forming an at least one channel that extends from the anterior portion of the body to the posterior portion of the body.

3. The fragrance packaging of claim 1 wherein the at least one channel is formed to allow for a valve arrangement to pass through the at least one channel.

4. The fragrance packaging of claim 3 wherein the valve arrangement comprises a sealing member arranged to seal a portion of at least one of the inlet or outlet of the shell, wherein the sealing member restricts an airflow through the at least one fragrance chamber.

5. The fragrance packaging of claim 1 wherein the scent medium is a natural ingredient or a synthetic ingredient.

6. The fragrance packaging of claim 5 wherein the natural ingredient or the synthetic ingredient is disposed on a substrate.

7. The fragrance packaging of claim 6 wherein the substrate comprises a perforated or grill structure.

8. The fragrance packaging of claim 6 wherein the substrate is an organic material.

9. The fragrance packaging of claim 8 wherein organic material is at least one of the group of:
paper,
fabric,
leaf,
petal,
seed,
seed pod,
flower, or
bark.

10. The fragrance packaging of claim 1 wherein the shell is comprised of a polymer or a glass.

11. The fragrance packaging of claim 10 wherein the shell is substantially transparent.

12. The fragrance packaging of claim 11 wherein the polymer or the glass form a plurality of windows in the shell.

13. The fragrance packaging of claim 1 wherein the shell is comprised of a porous material.

14. The fragrance packaging of claim 13 wherein the porous material is paper or a polymer.

15. A fragrance packaging for a scent dispensing system, the packaging comprising:
a body with an anterior portion and a posterior portion, the body adapted to fit a cartridge chamber in the scent dispensing system,
the body comprising a porous material,
the body forming a toroidal shell wherein an at least one channel is formed through the body parallel to an axis extending from the anterior portion to the posterior portion,
the toroid shell substantially enclosing an at least one fragrance chamber,
wherein the at least one fragrance chamber comprises the at least one scent medium, and
wherein the at least one scent medium is unable to pass from the at least one fragrance chamber to the cartridge chamber.

16. A method for packaging an at least one scent medium for a scent dispensing system, the method comprising:
forming a shell having an anterior portion and a posterior portion, wherein an interior of the shell substantially comprises an at least one fragrance chamber comprising the at least one scent medium
forming an inlet at the anterior portion and an outlet at the posterior portion,
wherein the inlet and the outlet comprise a plurality of apertures, and,
wherein the fragrance chamber is in fluid connection with the inlet and the outlet,
wherein the at least one scent medium is unable to pass from the fragrance chamber through the inlet or the outlet.
